(19) United States
(12) Patent Application Publication
(10) Pub. No.: US 2007/0299332 A1
(43) Pub. Date: Dec. 27, 2007

(54) IMAGING APPARATUS AND IMAGING METHOD

(76) Inventor: Hitoshi Ikeda, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 11/768,545

(22) Filed: Jun. 26, 2007

(30) Foreign Application Priority Data
Jun. 27, 2006 (JP) 2006-176653

(51) Int. Cl. A61B 5/055

(52) U.S. Cl. 600/410

(57) ABSTRACT

With the objective of improving diagnostic efficiency, image correction processing is effected on an actual scan image after the generation of the actual scan image to thereby generate a corrected image. Then, the corrected image is displayed on a display screen. When a control signal for displaying the pre-correction actual scan image is outputted based on a command issued from an operator here, the corrected image displayed on the display screen is displayed by switching to the pre-correction actual scan image.

### CROSS REFERENCE TO RELATED APPLICATIONS

This application claims the benefit of Japanese Application No. 2006-176653 filed Jun. 27, 2006.

### BACKGROUND OF THE INVENTION

The present invention relates to an imaging apparatus and an imaging method, and particularly to an imaging apparatus and an imaging method which effect image correction processing on a generated image to thereby generate a corrected image and thereafter display the corrected image on a display screen.

An imaging apparatus such as a magnetic resonance imaging (MRI) apparatus, an ultrasonic diagnostic apparatus, an X-ray CT apparatus or the like has frequently been used particularly in medical applications as a apparatus which generates a tomographic image about a tomographic plane of a subject.

For example, the magnetic resonance imaging apparatus photographs an image about a tomographic plane of a subject using a nuclear magnetic resonance (NMR) phenomenon. Described specifically, the subject is accommodated in a static magnetic field space to align spins of proton of the subject in a static magnetic field direction, thereby generating magnetization vectors. Then, an RF pulse having a resonant frequency is applied to generate a nuclear magnetic resonance phenomenon, thereby changing the magnetization vectors of the proton. Thereafter, the magnetic resonance imaging apparatus receives a magnetic resonance (MR) signal generated when the proton is returned to its original magnetization vector state, and generates, by image reconstruction, a tomographic image about a tomographic plane of the subject, based on the received magnetic resonance signal.

As an RF receiving coil for receiving the magnetic resonance signal in the magnetic resonance imaging apparatus, a surface coil such as a phased array coil or the like has frequently been used. However, the surface coil has such a characteristic that receiving sensitivity is reduced with distance from a source of generation of the magnetic resonance signal in the subject. A sensitivity distribution in the entire imaging area is not uniform spatially. Therefore, there is a case in which a sensitivity distribution at the whole imaging area is not uniform spatially.

There is, for example, a case in which a high frequency magnetic field formed by transmitting an RF pulse by means of an RF transmitting coil such as a body coil might be ununiform due to a dielectric constant effect upon imaging a subject in a high static magnetic field space having a magnetic field strength of 3 Teslas or higher.

Therefore, there is a case in which due to the fact that a reception sensitivity distribution and a transmission sensitivity distribution are spatially ununiform, artifacts occur in a tomographic image and image quality is deteriorated.

In order to cope with such problems, the tomographic image is subjected to image correction processing using the reception sensitivity distribution and the transmission sensitivity distribution. Described specifically, a reference image is acquired by executing a reference scan in addition to an actual scan, and a reception sensitivity distribution in the imaging area of the surface coil is measured using the reference image. A transmission sensitivity distribution is measured by, for example, a Double flip angle method. Thereafter, an actual scan image generated as a tomographic image by the actual scan is subjected to image correction processing using the measured reception sensitivity distribution and transmission sensitivity distribution to thereby generate a corrected image (refer to, for example, a patent document 1, a non-patent document 1 and a non-patent document 2).

[Patent Document 1] Japanese Unexamined Patent Publication No. 2005-177240 [Non-patent Document 1] Hiroaki Mihara et. al., A method of RF inhomogeneity correction in MR imaging, Magnetic Resonance Materials in Physics, Biology and Medicine 7, USA., 1998, p 115-p 120 [Non-patent Document 2] Jinghua Wang et. al., In vivo method for correcting transmit/receive nonuniformities with phased array coils, Magnetic Resonance in Medicine 53, USA., 2005, p 666-p 674

However, when such image correction processing is carried out upon image reconstruction, a corrected image generated by the image correction processing is displayed on a display screen and a pre-correction tomographic image is not displayed on the display screen. Therefore, there is a case in which since the pre-correction tomographic image is not displayed even when it is confirmed that the corrected image has been subjected to overcorrection, for example, an image diagnosis cannot be done with ease using the pre-correction tomographic image. There are cases in which since both of the tomographic image prior to the image correction processing and the corrected image subsequent to the image correction processing are generated upon reconstruction to display the tomographic image prior to the image correction processing, the amount of data increases and there is a need to increase storage capacity of a memory device which stores an image therein, and the operation of storing data by an operator becomes curbersome. Thus, this can cause a reduction in diagnostic efficiency.

### SUMMARY OF THE INVENTION

It is desirable that the problem described previously is solved.

There is provided an imaging apparatus of one aspect of the invention, comprising an image generating unit that generates an image, an image correcting unit that effects image correction processing on the image generated by the image generating unit to thereby generate a corrected image, and a display unit that displays the corrected image generated by the image correcting unit on a display screen, wherein the imaging apparatus includes a controller that outputs a control signal for causing the display unit to display the image generated by the image generating unit to the display unit and wherein when the display unit receives the control signal from the controller, the display unit displays the corrected image displayed on the display screen by switching to the image generated by the image generating unit.

There is provided an imaging method of another aspect of the invention, comprising: an image generating step for generating an image, an image correction processing step for performing image correction processing on the image generated at the image generating step to thereby generate a corrected image, and a display step for displaying the corrected image generated at the image correction processing step on a display screen, wherein the imaging method includes a control step for outputting a control signal for displaying the image generated at the image generating step at the display step and wherein at the display step, the corrected image displayed on the display screen is displayed by switching to the image generated at the image generating step when the control signal outputted at the control step is received.

According to the invention, an imaging apparatus and an imaging method capable of improving diagnostic efficiency can be provided.

Further objects and advantages of the present invention will be apparent from the following description of the preferred embodiments of the invention as illustrated in the accompanying drawings.

### BRIEF DESCRIPTION OF THE DRAWINGS

FIGS. 1(a) and 1(b) are configurational diagrams showing a construction of a magnetic resonance imaging apparatus 1 illustrative of an embodiment according to the invention.

FIG. 2 is a flow chart showing operation of the magnetic resonance imaging apparatus 1 illustrative of the embodiment according to the invention.

FIG. 3 is a diagram showing the flow of data at the time that an imaging area of a subject SU is photographed by the magnetic resonance imaging apparatus 1 illustrative of the embodiment according to the invention.

FIGS. 4(a), 4(b), and 4(c) are diagrams showing images displayed by the magnetic resonance imaging apparatus 1 and illustrative of the embodiment according to the invention.

FIG. 5 is a diagram showing the relationship between a deviation σ_{f} of a transmission sensitivity distribution f(x, y) and an actually measured distribution of flip angles θ in the embodiment according to the invention.

### DETAILED DESCRIPTION OF THE INVENTION

One example illustrative of an embodiment according to the invention will hereinafter be explained with reference to the accompanying drawings.

(Apparatus Construction)

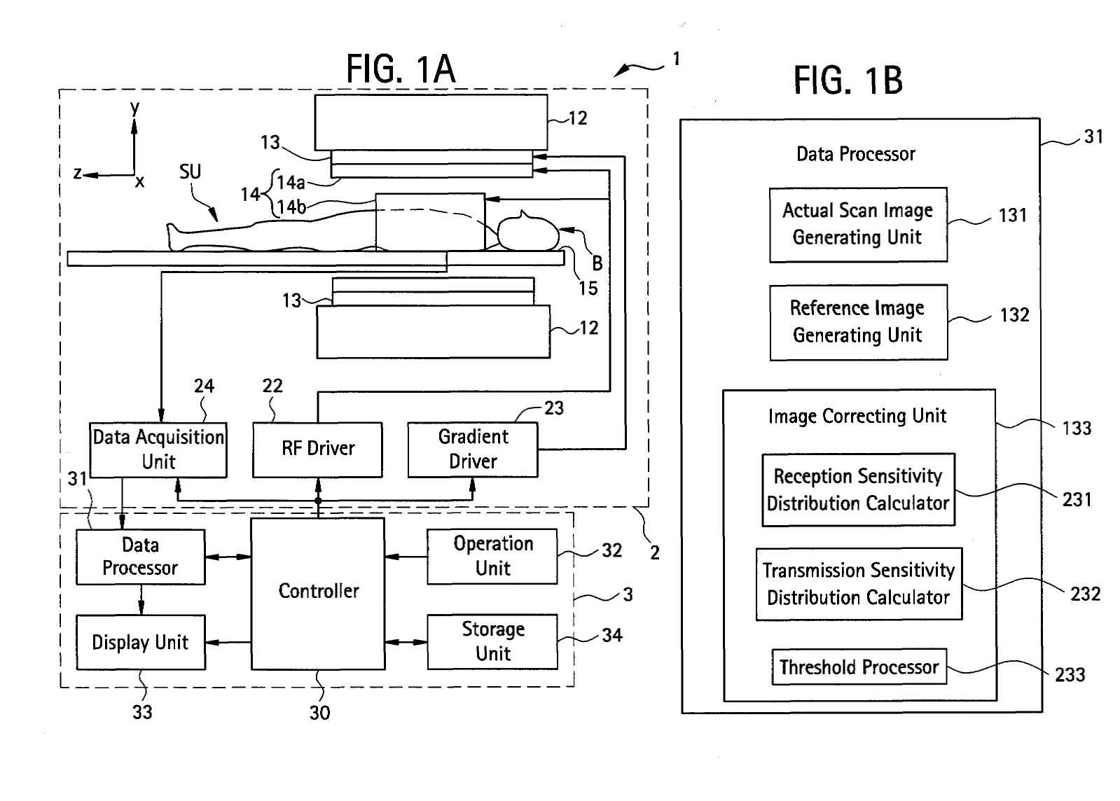

FIG. 1 is a configurational diagram showing a construction of a magnetic resonance imaging apparatus 1 illustrative of the embodiment according to the invention. In FIG. 1, FIG. 1(a) is a configurational diagram typically showing an overall construction of the magnetic resonance imaging apparatus 1. FIG. 1(b) is a block diagram showing a construction of a data processor 31 included in the overall construction of the magnetic resonance imaging apparatus 1.

As shown in FIG. 1(a), the magnetic resonance imaging apparatus 1 showing the present embodiment has a scan section 2 and an operation console section 3.

The scan section 2 will be described.

As shown in FIG. 1(a), the scan section 2 has a static magnetic field magnet unit 12, a gradient coil unit 13, an RF coil part 14, a cradle 15, an RF driver 22, a gradient driver 23 and a data acquisition unit 24. The scan section 2 executes an actual scan AS for transmitting an RF pulse to a subject SU so as to excite the spin of the subject SU in an imaging space B formed with a static magnetic field and transmitting a gradient pulse to the subject SU to which the RF pulse has been transmitted, thereby obtaining a magnetic resonance signal generated in the subject SU as actual scan data. Further, the scan section 2 effects a reference scan RS on the subject SU before execution of the actual scan AS to thereby acquire a magnetic resonance signal generated by the reference scan RS as reference scan data.

Respective constituent elements of the scan section 2 will be explained sequentially.

The static magnetic field magnet unit 12 comprises, for example, a superconductive magnet (not shown) and forms a static magnetic field in the imaging space B in which the subject SU is accommodated or held. Here, the static magnetic field magnet unit 12 forms the static magnetic field so as to extend along a body-axis direction (z direction) of the subject SU placed on the cradle 15. Incidentally, the static magnetic field magnet unit 12 may be constituted of a pair of permanent magnets.

The gradient coil unit 13 forms a gradient magnetic field in the imaging space B formed with the static magnetic field and applies or adds spatial position information to the magnetic resonance signal received by the RF coil part 14. Here, the gradient coil unit 13 comprises three systems set so as to correspond to three-axis directions of a z direction extending along a static magnetic field direction and x and y directions orthogonal to the z direction one another. They transmit gradient pulses in such a manner that a gradient magnetic field is formed in each of a frequency encode direction, a phase encode direction and a slice selection direction according to an imaging condition. Described specifically, the gradient coil unit 13 applies the gradient magnetic field in the slice selection direction of the subject SU and selects a slice of the subject SU excited by transmission of the RF pulse by the RF coil part 4. The gradient coil unit 13 applies the gradient magnetic field in the phase encode direction of the subject SU and phase-encodes a magnetic resonance signal from the slice excited by the RF pulse. And the gradient coil unit 13 applies the gradient magnetic field in the frequency encode direction of the subject SU and frequency-encodes the magnetic resonance signal from the slice excited by the RF pulse.

The RF coil part 14 transmits the RF pulse corresponding to an electromagnetic wave to its corresponding imaging area of the subject SU within the imaging space B formed with the static magnetic field by the static magnetic field magnet unit 12 to form a high frequency magnetic field, thereby exciting the spins of proton in the imaging area of the subject SU. The RF coil part 14 receives an electromagnetic wave generated from the excited proton in the imaging area of the subject SU as a magnetic resonance signal. In the present embodiment, the RF coil part 14 has a first RF coil 14a and a second RF coil 14b as shown in FIG. 1(a). Here, the first RF coil 14a is of, for example, a bird cage type body coil and is disposed so as to surround the imaging area of the subject SU. On the other hand, the second RF coil 14b is of a coil ununiform in reception sensitivity distribution as compared with the first RF coil 14a in its imaging area. The second RF coil 14b is a phased array coil and has a plurality of surface coils disposed along the surface of the imaging area of the subject SU.

The cradle 15 has a table that places the subject SU thereon. The cradle 15 moves the table between the inside and outside of the imaging space B, based on a control signal supplied from a controller 30.

The RF driver 22 drives the RF coil part 14 to transmit an RF pulse to within the imaging space B, thereby forming a high frequency magnetic field in the imaging space B. The RF driver 22 modulates a signal sent from an RF oscillator (not shown) to a signal having predetermined timing and predetermined envelope using a gate modulator (not shown) on the basis of the control signal outputted from the controller 30. Thereafter, the RF driver 22 allows an RF power amplifier (not shown) to amplify the signal modulated by the gate modulator and outputs the same to the RF coil part 14, and allows the RF coil part 14 to transmit the RF pulse.

The gradient driver 23 applies a gradient pulse to the gradient coil unit 13 based on the control signal outputted from the controller 30 to drive the gradient coil unit 13, thereby to generate a gradient magnetic field within the imaging space B formed with the static magnetic field. The gradient driver 23 has a three-system drive circuit (not shown) in association with the three-system gradient coil unit 13.

The data acquisition unit 24 acquires a magnetic resonance signal received by the RF coil part 14 based on the control signal outputted from the controller 30. Here, the data acquisition unit 24 phase-detects the magnetic resonance signal received by the RF coil part 14 using a phase detector (not shown) with the output of the RF oscillator (not shown) of the RF driver 22 as a reference signal. Thereafter, the data acquisition unit 24 converts the magnetic resonance signal corresponding to the analog signal into a digital signal by using an A/D converter (not shown) and outputs it therefrom.

The operation console section 3 will be explained.

As shown in FIG. 1(a), the operation console section 3 has the controller 30, a data processor 31, an operation unit 32, a display or display unit 33 and a storage unit 34.

Respective constituent elements of the operation console section 3 will be described sequentially.

The controller 30 has a computer and a memory that stores programs that allow the computer to execute predetermined data processing and controls respective parts. Here, the controller 30 receives operation data sent from the operation unit 32 and outputs the control signal to the RF driver 22, gradient driver 23 and data acquisition unit 24 respectively, based on the operation data inputted from the operation unit 32, thereby executing a predetermined scan. Along with it, the controller 30 outputs control signals to the data processor 31, display unit 33 and storage unit 34 to perform control on the respective parts. In the present embodiment, although described later for detail, the controller 30 outputs a control signal for allowing the display unit 33 to display an actual scan image generated by an actual scan image generating unit 131 of the data processor 31 to be described later, to the display unit 33 based on a command inputted to the operation unit 32 by an operator.

The data processor 31 has a computer and a memory which stores programs that execute predetermined data processing using the computer. The data processor 31 generates an image, based on the control signal supplied from the controller 30. Here, the data processor 31 uses the magnetic resonance signal obtained by executing a scan by the scan section 2 as row data and reconstructs the image about the subject SU. Then, the data processor 31 outputs the generated image to the display 33.

As shown in FIG. 1(b), the data processor 31 has the actual scan image generating unit 131, a reference image generating unit 132 and an image correcting unit 133.

Here, the actual scan image generating unit 131 uses a magnetic resonance signal obtained by performing an actual scan on the imaging area of the subject SU as row data and thereby generates an actual scan image about the imaging area of the subject SU.

The reference image generating unit 132 uses a magnetic resonance signal obtained by a reference scan executed prior to the actual scan about the imaging area of the subject SU as row data and thereby generates a reference scan image about the imaging area of the subject SU.

The image correcting unit 133 effects image correction processing on the actual scan image generated by the actual scan image generating unit 131 as a tomographic image to thereby generate a corrected image.

As shown in FIG. 1(b), the image correcting unit 133 has a reception sensitivity distribution calculator 231, a transmission sensitivity distribution calculator 232 and a threshold processor 233. Here, the reception sensitivity distribution calculator 231 calculates a reception sensitivity distribution at the time that the RF coil part 14 receives the magnetic resonance signal therein, in the imaging area of the subject SU. The transmission sensitivity distribution calculator 232 calculates a transmission sensitivity distribution at the time that the RF coil part 14 transmits the RF pulse, in the imaging area of the subject SU. The threshold processor 233 effects threshold processing on the transmission sensitivity distribution calculated by the transmission sensitivity distribution calculator 232. The image correcting unit 133 effects image correction processing on the actual scan image, using the reception sensitivity distribution calculated by the reception sensitivity distribution calculator and the transmission sensitivity distribution threshold-processed by the threshold processor 233.

The data processor 31 is constructed as described above.

The operation unit 32 is constituted of an operation device such as a keyboard, a pointing device or the like. An operator inputs operation data to the operation unit 32, and the operation unit 32 outputs the operation data to the controller 30. In the present embodiment, the operation unit 32 receives a command for causing the display unit 33 to display the actual scan image generated by the actual scan image generating unit 131, by means of the operation of the operator and outputs its operation data to the controller 30.

The display unit 33 is constituted of a display device such as a CRT and displays an image on its display screen, based on the control signal outputted from the controller 30. For example, the display unit 33 displays images about input items corresponding to the operation data inputted to the operation unit 32 by the operator on the display screen in plural form. Further, the display unit 33 receives data about the image of the subject SU generated based on the magnetic resonance signal from the subject SU from the data processor 31 and displays the image on the display screen. In the present embodiment, the display unit 33 first displays the corrected image generated by the image correcting unit 133 on the display screen. Then, the command for allowing the display unit 33 to display the actual scan image generated by the actual scan image generating unit 131 is inputted to the operation unit 32 by the operation of the operator. The controller 30 outputs the corresponding control signal for causing the display unit 33 to display the actual scan image to the display unit 33. When the display unit 33 receives the control signal therein, the display unit 33 displays the corrected image displayed on the display screen by switching to the actual scan image generated by the actual scan image generating unit 131. That is, when the display unit 33 receives the control signal from the controller 30, the display unit 33 receives the actual scan image stored in the storage unit 34 and displays it on the display screen.

The storage unit 34 comprises a memory and stores various data therein. In the storage unit 34, the stored data are accessed by the controller 30 as needed. In the present embodiment, the storage unit 34 receives the actual scan image from the actual scan image generating unit 131 and stores the actual scan image generated by the actual scan image generating unit 131.

(Operation)

A description will be made below of the operation of the magnetic resonance imaging apparatus 1 showing the embodiment according to the invention.

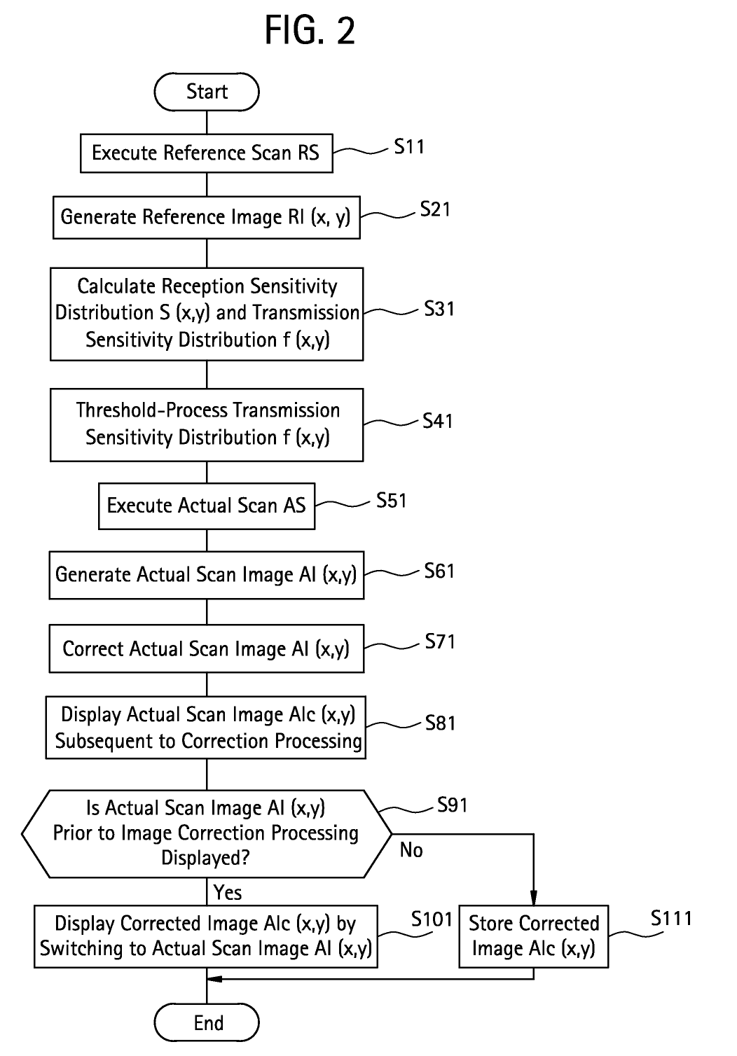

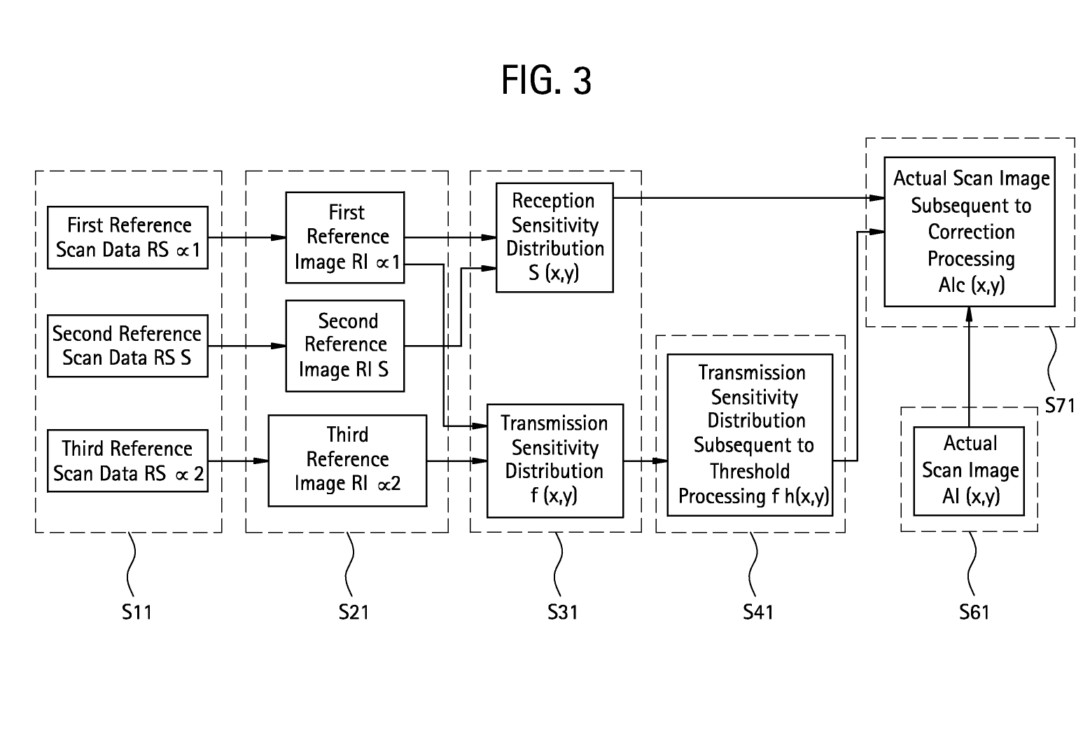

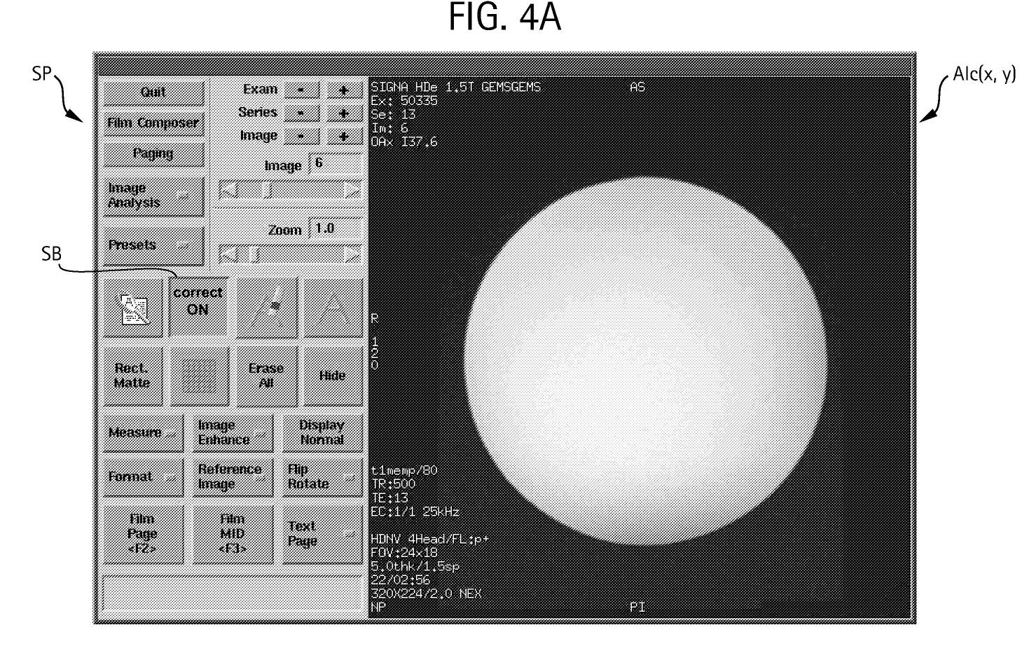

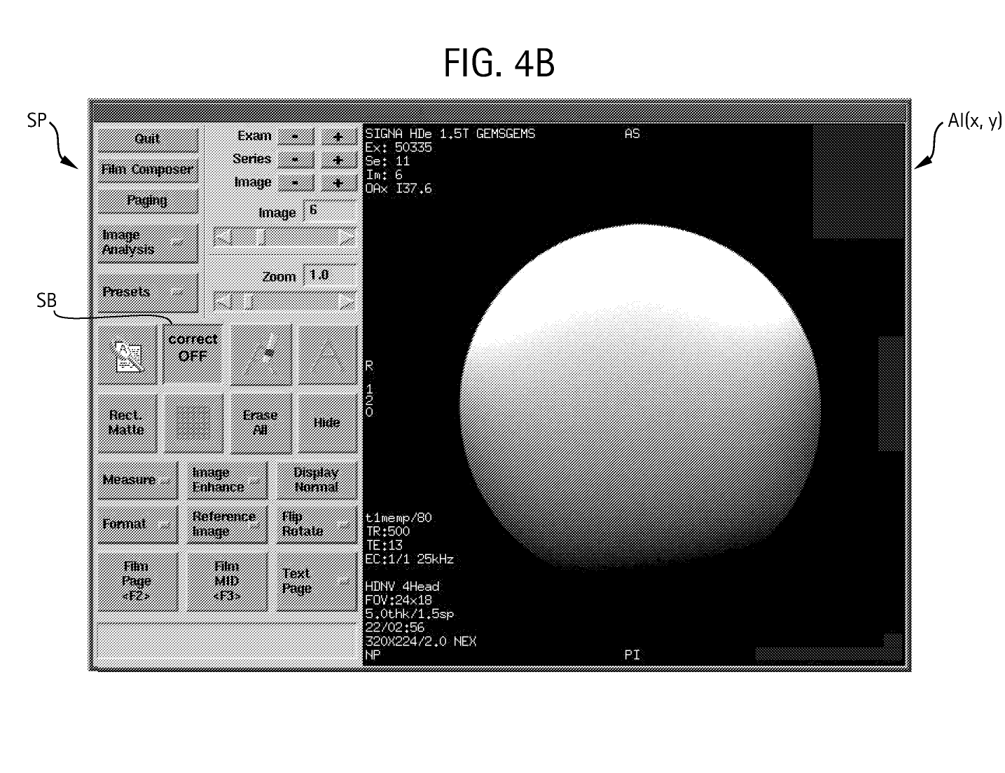

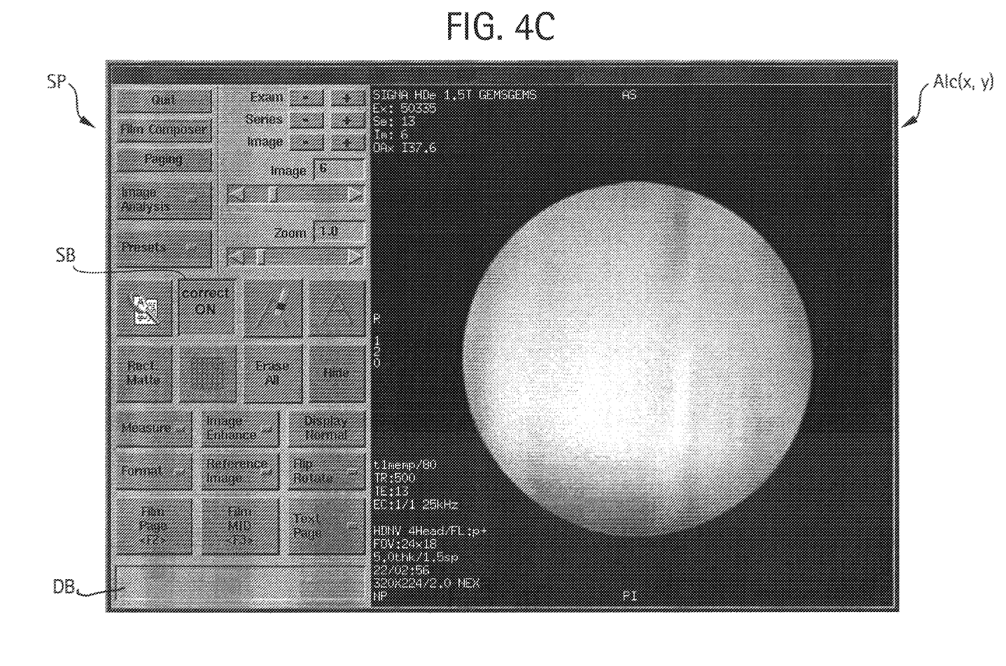

FIG. 2 is a flow chart showing the operation of the magnetic resonance imaging apparatus 1 illustrative of the embodiment according to the invention. FIG. 3 is a diagram showing the flow of data at the time that an imaging area of a subject SU is photographed by the magnetic resonance imaging apparatus 1 illustrative of the embodiment according to the invention. FIG. 4 is a diagram showing images displayed by the magnetic resonance imaging apparatus 1 illustrative of the embodiment according to the invention.

As shown in FIG. 2, a reference scan RS is first executed (S11).

Here, the scan section 2 executes the reference scan RS for allowing the RF coil part 14 to transmit an RF pulse to the imaging area of the subject SU photographed by the actual scan AS and allowing the RF coil part 14 to receive a magnetic resonance signal generated in the imaging area of the subject SU.

In the present embodiment, the scan section 2 executes a first reference scan RS1, a second reference scan RS2 and a third reference scan RS3 respectively as the reference scan RS. Here, the first reference scan RS1, the second reference scan RS2 and the third reference scan RS3 are respectively executed by a gradient echo method.

Described specifically, the scan section 2 executes the first reference scan RS1 in such a manner that the first RF coil 14a corresponding to the body coil transmits an RF pulse of a first flip angle α1 to the imaging area of the subject SU, and the first RF coil 14a receives a magnetic resonance signal generated in the imaging area therein. The magnetic resonance signal obtained by the execution of the first reference scan RS1 is acquired as first reference scan data RS_{α1}.

The scan section 2 executes the second reference scan RS2 in such a manner that the first RF coil 14a corresponding to the body coil transmits the RF pulse of the first flip angle α1 to the imaging area of the subject SU, and the second RF coil 14b corresponding to the phased array coil receives a magnetic resonance signal generated in the imaging area. The magnetic resonance signal obtained by the execution of the second reference scan RS2 is acquired as second reference scan data RSs.

The scan section 2 executes the third reference scan RS3 in such a manner that the first RF coil 14a corresponding to the body coil transmits an RF pulse of a second flip angle α2 different from the first flip angle α1 to the imaging area of the subject SU, and the first RF coil 14a receives a magnetic resonance signal generated in the imaging area. In the present embodiment, upon execution of the third reference scan RS3, the first RF coil 14a transmits the RF pulse to the imaging area in such a manner that the second flip angle α2 reaches one-half of the first flip angle α1. The magnetic resonance signal obtained by the execution of the third reference scan RS3 is acquired as third reference scan data RSC_{α2}. Incidentally, since a computational equation can be simplified as expressed in an equation (2) to be described later by setting the second flip angle α2 to one-half of the first flip angle α1, data processing at the calculation of a B1 distribution θ (x, y) can be speeded up.

Thus, the first reference scan data RS_{α1}, the second reference scan data RSs and the third reference scan data RS_{α2} are respectively acquired in the actual Step (S11) as shown in FIG. 3.

Next, as shown in FIG. 2, the generation of a reference image R1 (x, y) is executed (S21).

Here, the reference image generating unit 132 generates the reference image R1 (x, y) about the imaging area, based on the magnetic resonance signal obtained by the execution of the reference scan RS. In the present embodiment, a first reference image RI_{α1} (x, y), a second reference image RIs (x, y) and a third reference image RI_{α2} (x, y) are respectively generated as the reference image RI (x, y).

Described specifically, as shown in FIG. 3, the reference image generating unit 132 generates the first reference image RI_{α1} (x, y) about the imaging area of the subject SU, based on the first reference scan data RS_{α1} obtained by the execution of the first reference scan RS1.

As shown in FIG. 3, the reference image generating unit 132 generates the second reference image RIs (x, y) about the imaging area of the subject SU, based on the second reference scan data RSs obtained by the execution of the second reference scan RS2.

As shown in FIG. 3, the reference image generating unit 132 generates the third reference image RI_{α2} (x, y) about the imaging area of the subject SU, based on the third reference scan data RS_{α2} obtained by the execution of the third reference scan RS3.

Next, as shown in FIG. 2, the calculation of a reception sensitivity distribution S (x, y) and a transmission sensitivity distribution f (x, y) is carried out (S31).

Here, the reception sensitivity distribution calculator 231 calculates the reception sensitivity distribution S (x, y), based on the first reference image RI_{α1} (x, y) and the second reference image RIs (x, y) as shown in FIG. 3.

Described specifically, respective pixel data of the first reference image RI_{α1} (x, y) are divided by respective pixel data of the second reference image RIs (x, y) by means of the reception sensitivity distribution calculator 231 to calculate a reception sensitivity distribution S (x, y) as expressed in the following equation (1):

$$\begin{matrix}[ Equation 1 ] & \\ s ( x , y ) = \frac{{RI}_{α 1} ( x , y )}{{RI}_{s} ( x , y )} & ( 1 )\end{matrix}$$

On the other hand, as to the transmission sensitivity distribution f (x, y), the transmission sensitivity distribution (transmission sensitivity non-uniformity distribution) f (x, y) developed in the actual scan image generated by the actual scan AS is calculated based on the first reference image RI_{α1} (x, y) and the third reference image RI_{α2} (x, y) as shown in FIG. 3. Here, the transmission sensitivity distribution calculator 232 calculates a B1 distribution (flip angle distribution) θ (x, y) using the first reference image RI_{α1} (x, y) and the third reference image RI_{α2} (x, y) and thereafter calculates the transmission sensitivity distribution (transmission sensitivity non-uniformity distribution) f(x, y) developed in the actual scan image by the actual scan SA, based on the B1 distribution.

Described specifically, a B1 distribution θ (x, y) about the imaging area of the subject SU is calculated using the first reference image RI_{α1} (x, y) and the third reference image RI_{α2} (x, y) as expressed in the following equation (2):

$$\begin{matrix}[ Equation 2 ] & \\ θ ( x , y ) = 2 {cos}^{- 1} ( \frac{{RI}_{α 1} ( x , y )}{2 {RI}_{α 2} ( x , y )} ) & ( 2 )\end{matrix}$$

Then, a transmission sensitivity distribution f (x, y) related to an actual scan image generated by executing the actual scan AS in a spin echo sequence is calculated as expressed in the following equation (3):

$$\begin{matrix}[ Equation 3 ] & \\ f ( x , y ) = \frac{α}{θ ( x , y ) {sin}^{3} ( \frac{π}{2} \frac{θ ( x , y )}{α} )} & ( 3 )\end{matrix}$$

On the other hand, a transmission sensitivity distribution f (x, y) related to an actual scan image generated by executing the actual scan AS in a gradient echo sequence is calculated as expressed in the following equation (4):

$$\begin{matrix}[ Equation 4 ] & \\ f ( x , y ) ) = \frac{α sin ( β )}{θ ( x , y ) sin ( β \frac{θ ( x , y )}{α} )} & ( 4 )\end{matrix}$$

Incidentally, in the above equations (3) and (4), α indicates a flip angle at the time that the first reference scan is carried out, and β indicates a flip angle at the time that the actual scan AS is carried out in the gradient echo sequence.

Next, as shown in FIG. 2, threshold processing on the transmission sensitivity distribution f (x, y) is executed (S41).

Here, as shown in FIG. 3, the threshold processor 233 effects threshold processing on the transmission sensitivity distribution f (x, y) calculated by the transmission sensitivity distribution calculator 232 and outputs a transmission sensitivity distribution fh (x, y) subsequent to its threshold processing.

Described specifically, a partial differential value (∂f (x, y)/∂RI_{α1} (x, y)) of the transmission sensitivity distribution f (x, y) and the first reference image RI_{α1} (x, y), and a partial differential value (∂f (x, y)/∂RI_{α2} (x, Y)) of the transmission sensitivity distribution f (x, y) and the third reference image RI_{α2} (x, y) are respectively calculated. Further, a deviation σRI_{α1} of the first reference image RI_{α1} (x, y) and a deviation σRI_{α1} of the third reference image RI_{α2} (x, y) are calculated.

Thereafter, as expressed in the following equation (5), a partial differential value (∂f (x, y)/∂RI_{α1} (x, y)) of the transmission sensitivity distribution f (x, y) and the first reference image RI_{α1} (x, y), and a partial differential value (∂f (x, y)/∂RI_{α2} (x, y)) of the transmission sensitivity distribution f (x, y) and the third reference image RI_{α2} (x, y) are respectively calculated. Further, a deviation σ_{f} of the transmission sensitivity distribution f (x, y) is calculated using the deviation σRI_{α1} of the first reference image RI_{α1} (x, y) and the deviation σRI_{α1} of the third reference image RI_{α2} (x, y).

$$\begin{matrix}[ Equation 5 ] & \\ {σ}_{f}^{2} = {( \frac{\partial f ( x , y )}{\partial {RI}_{α 1} ( x , y )} )}^{2} {σ}_{{RI}_{α 1}}^{2} + {( \frac{\partial f ( x , y )}{\partial {RI}_{α 2} ( x , y )} )}^{2} {σ}_{{RI}_{α 2}}^{2} & ( 5 )\end{matrix}$$

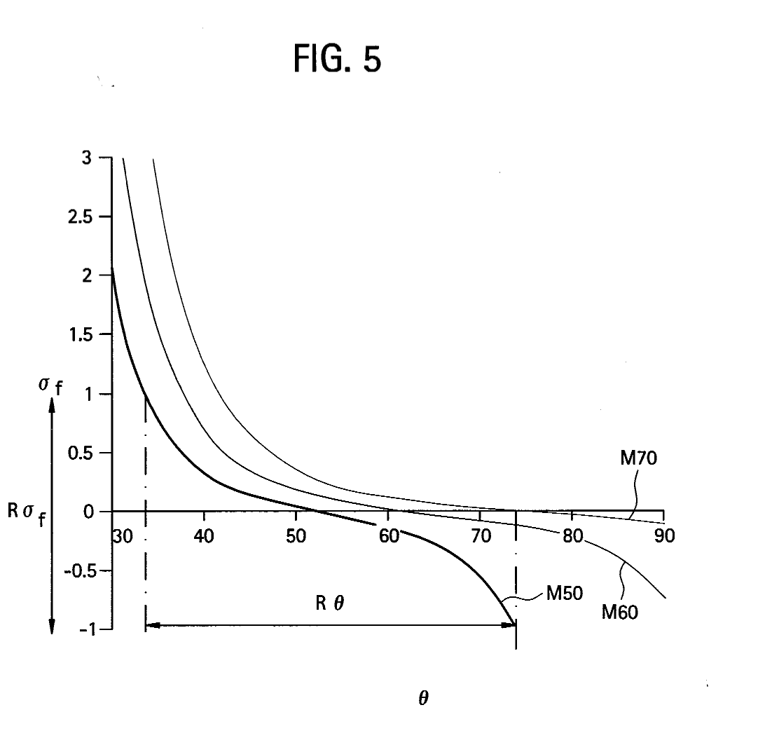

FIG. 5 is a diagram showing the relationship between a deviation σ_{f} of a transmission sensitivity distribution f(x, y) and an actually measured distribution of flip angles θ in the embodiment according to the invention. When predetermined flip angles are set, the relationship between the actually measured distribution of flip angles θ and the deviation σ_{f} of the transmission sensitivity distribution f (x, y) is shown in FIG. 5. Here, results obtained at the time that the flip angles are set as 50°, 60° and 70° are illustrated as M50, M60 and M70 respectively.

A range Rθ of flip angles θ corresponding to a deviation range R_{σf} set in advance at the deviation σ_{f} of the transmission sensitivity distribution f (x, y) is calculated from the relationship between the deviation σ_{f} of the transmission sensitivity distribution f (x, y) calculated as shown in FIG. 5 and the actually measured distribution of flip angles θ. When the pre-set deviation range R_{σf} ranges from −1.0 to 1.0 upon setting the flip angle to 50° as shown in FIG. 5 by way of example, it is determined that the range Rθ of the flip angles θ corresponding to it extends from 33° to 75°.

A range Rf of a transmission sensitivity distribution f (x, y) associated with the determined range Rθ of flip angles θ is determined using the equations (3) and (4), and the range thereof is set as a threshold value.

Thereafter, the transmission sensitivity distribution f (x, y) is threshold-processed using the set threshold value, and a transmission sensitivity distribution fh (x, y) subsequent to the threshold processing is outputted. That is, data lying within a range corresponding to the threshold values at the transmission sensitivity distribution f (x, y) is outputted as the transmission sensitivity distribution fh (x, y) subsequent to the threshold processing. By executing the threshold processing in this way, a portion large in deviation at the transmission sensitivity distribution f (x, y) is removed and the transmission sensitivity distribution is processed within a small deviation range.

Incidentally, since the portion removed or taken out by the threshold processing is indefinite in transmission sensitivity distribution, data is extrapolated by a local polynomial approximation using the values of portions located close to one another at the transmission sensitivity distribution fh (x, y). The extrapolated data is processed by a low-pass filter.

The execution of the actual scan AS is next done as shown in FIG. 2 (S51).

Here, the RF coil part 14 transmits an RF pulse to the imaging area of the subject SU in the imaging space B formed with the static magnetic field and receives a magnetic resonance signal generated in the imaging area to which the RF pulse has been transmitted, as actual scan data, whereby the actual scan AS is carried out. The actual scan AS is performed in accordance with, for example, a pulse sequence such as the spin echo sequence or gradient echo sequence.

Next, the generation of an actual scan image AI (x, y) is done as shown in FIG. 2 (S61).

Here, the magnetic resonance signal obtained as the actual scan data by the execution of the actual scan AS is set as row data, and the actual scan image AI (x, y) about its imaging area is produced by the actual scan image generating unit 131. Then, data about the actual scan image is outputted from the actual scan image generating unit 131 to the storage unit 34, and the data about the actual scan image is stored in the storage unit 34.

As shown in FIG. 2, the actual scan image AI (x, y) is next corrected (S71).

Here, as shown in FIG. 3, the image correcting unit 133 performs image correction processing on the actual scan image AI (x, y) generated by the actual scan image generating unit 131, using the reception sensitivity distribution S (x, y) and the transmission sensitivity distribution fh (x, y) subsequent to the threshold processing.

Described specifically, as expressed in the following equation (6), the reception sensitivity distribution S (x, y) and the transmission sensitivity distribution fh (x, y) subsequent to the threshold processing are respectively integrated or accumulated with respect to the actual scan image AI (x, y) for every pixel of each individual position as viewed in x and y directions at the actual scan image AI (x, y), whereby image correction processing is effected on the actual scan image AI (x, y) to produce a corrected image AIc (x, y).

AI_{c}(x, y)=AI(x, y)×fh(x, y)×s(x, y) (6)

Next, the corrected image AIc (x, y) is displayed as shown in FIG. 2 (S81).

Here, the display unit 33 displays the corrected image AIc (x, y) produced by executing image correction processing by the image correcting unit 133 on its display screen.

FIG. 4(a) is a diagram showing a display screen which displays a corrected image AIc (x, y) by the display unit 33 in the embodiment according to the invention.

As shown in FIG. 4(a), a corrected image AIc (x, y) generated by execution of image correction processing and an operation or control panel image SP in which a plurality of operation items are arranged, are displayed on the display screen. The operation panel image SP includes an operation button SB indicating whether the image correction processing should be performed. At an actual step, the operation button SB is displayed as, for example, “correct. ON” so as to indicate that the image correction processing has been conducted.

Next, as shown in FIG. 2, it is determined whether an actual scan image AI (x, y) prior to the image correction processing should be displayed (Yes) or not (No) (S91).

Here, an operator observes the corrected image AIc (x, y) displayed on the display screen by the display unit 33 and makes a decision as to whether the actual scan image AI (x, y) prior to the image correction processing should be displayed. When it is confirmed that the corrected image AIc (x, y) has been overcorrected, for example, the actual scan image AI (x, y) is displayed (Yes). On the other hand, when it is not confirmed that the corrected image AIc (x, y) has been overcorrected, for example, the actual scan image AI (x, y) is not displayed (No).

When it is determined as shown in FIG. 2 that the actual scan image AI (x, y) prior to the image correction processing is displayed (Yes), the corrected image AIc (x, y) is displayed by switching to the actual scan image AI (x, y) (S101).

Here, a command for causing the display unit 33 to display an actual scan image generated by the actual scan image generating unit 131 is inputted to the operation unit 32 by operation of the operator. The controller 30 outputs a control signal for causing the display unit 33 to display the actual scan image to the display unit 33. When the display unit 33 receives the control signal, the display unit 33 switches the corrected image displayed on its display screen to the actual scan image generated by the actual scan image generating unit 131 and displays the same thereon. Described specifically, when the display unit 33 receives the control signal from the controller 30, the display unit 33 displays the actual scan image AI (x, y) on the display screen in response to data about the actual scan image AI (x, y) stored in the storage unit 34.

FIG. 4(b) is a diagram showing a display screen which displays an actual scan image AI (x, y) by the display unit 33 in the embodiment according to the invention.

As shown in FIG. 4(b), the actual scan image AI (x, y) is displayed at a position where the corrected image AIc (x, y) is displayed on the display screen. At this time, the operation button SB indicating whether the image correction processing should be performed is displayed as, for example, “correct. OFF” in the operation panel image SP so as to indicate that the image correction processing has not been conducted.

On the other hand, when it is determined as shown in FIG. 2 that the actual scan image AI (x, y) prior to the image correction processing is not displayed (No), the corrected image AIc (x, y) is stored (S111).

Here, the corrected image AIc (x, y) is stored in the storage unit 34 based on the command given from the operator. In the present embodiment, data about the pre-correction actual scan image AI (x, y) stored in the storage unit 34 is overwritten with data about the corrected image AIc (x, y), whereby the corrected image AIc (x, y) is stored.

FIG. 4(c) is a diagram showing a display screen displayed upon storage of a corrected image AIc (x, y) in the embodiment according to the invention.

As shown in FIG. 4(c), the operator inputs a command for storing the corrected image AIc (x, y) to a dialog box DB inputted with text data at the operation panel image SP, using the keyboard of the operation unit 32. For example, text data of “ps” is inputted. Thereafter, the controller 30 causes the storage unit 34 to store data about the corrected image AIc (x, y) based on the inputted command.

In the present embodiment as described above, the actual scan image AI (x, y) is subjected to the image correction processing after the generation of the actual scan image AI (x, y), whereby the corrected image AIc (x, y) is produced. Then, the corrected image AIc (x, y) is displayed on the display screen. Here, when the control signal for displaying the pre-correction actual scan image AI (x, y) is outputted based on the command issued from the operator, the corrected image AIc (x, y) displayed on the display screen is displayed by switching to the pre-correction actual scan image AI (x, y). Therefore, in the present embodiment, even when the corrected image generated by the image correction processing is displayed on the display screen where the image correction processing is performed upon image reconstruction, the pre-correction actual scan image can easily be displayed on the display screen. Thus, when it is confirmed that the image to be corrected has been overcorrected, the pre-correction actual scan image is displayed to make it easy to perform an image diagnosis. It is thus possible to enhance diagnostic efficiency.

The data about the pre-correction actual scan image AI (x, y) stored in the storage unit 34 is overwritten with the data about the corrected image AIc (x, y), whereby the corrected image AIc (x, y) is stored. Therefore, the operator is able to simplify the operation of storing the data. It is thus possible to improve diagnostic efficiency.

Incidentally, the invention is not limited to the above embodiment upon implementation of the invention. Various modifications can be adopted.

In the present embodiment, for example, the data about the pre-correction actual scan image AI (x, y) stored in the storage unit 34 is used to thereby display the corrected image AIc (x, y) displayed on the display screen by switching to the pre-correction actual scan image AI (x, y). However, the invention is not limited to it. For example, the corrected image AIc (x, y) is reverse-corrected to thereby generate data about a pre-correction actual scan image AI (x, y), after which the actual scan image AI (x, y) may be displayed using the data.

Although the present embodiment shows the case in which the sensitivity uniformity is corrected, the invention is not limited to it. The invention can be applied even in the case where processing such as smoothing processing or edge emphatic processing is carried out as the image correction processing.

Many widely different embodiments of the invention may be configured without departing from the sprit and the scope of the present invention. It should be understood that the present invention is not limited to the specific embodiments described in the specification, except as defined in the appended claims.

1. An imaging apparatus comprising:
an image generating unit that generates an image;
an image correcting unit that effects image correction processing on the image generated by the image generating unit to thereby generate a corrected image; and
a display unit that displays the corrected image generated by the image correcting unit on a display screen,
said imaging apparatus including a controller that outputs to the display unit a control signal for causing the display unit to display the image generated by the image generating unit,
wherein when the display unit receives the control signal from the controller, the display unit displays the corrected image displayed on the display screen by switching to the image generated by the image generating unit.

2. The imaging apparatus according to claim 1, further comprising a storage unit that stores the image generated by the image generating unit,
wherein when the display unit receives the control signal from the controller, the display unit displays the image stored in the storage unit.

3. The imaging apparatus according to claim 2, further comprising an operation unit to which a command for causing the display unit to display the image generated by the image generating unit is inputted by operation of an operator,
wherein the controller outputs the control signal, based on the command inputted to the operation unit by the operator.

4. The imaging apparatus according to claim 3, further comprising a scan section that transmits an RF pulse to a subject in a static magnetic filed space and performs a scan for receiving a magnetic resonance signal generated at the subject to which the RF pulse is transmitted,
wherein the image generating unit generates as the image a tomographic image about a tomographic plane of the subject, based on the magnetic resonance signal received by the scan section.

5. The imaging apparatus according to claim 4,
wherein the scan section executes reference scans and an actual scan.

6. The imaging apparatus according to claim 5,
wherein the reference scans comprises a first reference scan, a second reference scan and a third reference scan,
in the first reference scan, a first RF coil corresponding to a body coil transmits an RF pulse of a first flip angle to an imaging area of the subject, and the first RF coil receives a magnetic resonance signal generated in the imaging area therein,
in the second reference scan, the first RF coil transmits an RF pulse of the first flip angle to the imaging area of the subject, and a second RF coil corresponding to a phased array coil receives a magnetic resonance signal generated in the imaging area therein,
in the third reference scan, the first RF coil transmits an RF pulse of a second flip angle different from the first flip angle to the imaging area of the subject, and the first RF coil receives a magnetic resonance signal generated in the imaging area therein.

7. The imaging apparatus according to claim 6,
wherein a first reference image is generated from the first reference scan and a second reference image is generated from the second reference scan, and a reception sensitivity distribution is obtained based on the first reference image and the second reference image.

8. The imaging apparatus according to claim 7,
wherein a third reference image is generated from the third reference scan, and a transmission sensitivity distribution is obtained based on the first reference image and the second reference image.

9. The imaging apparatus according to claim 8,
wherein on the transmission sensitivity distribution threshold processing is executed.

10. The imaging apparatus according to claim 9,
wherein the corrected image AIc(x, y) is obtained based on the following equation that includes the reception sensitivity distribution S(x, y), the transmission sensitivity distribution fh(x, y) subsequent to the threshold processing and an actual scan image AI(x, y) that is generated from the actual scan.
AI_{c}(x, y)=AI(x, y)×fh(x, y)×s(x, y)

11. An imaging method comprising:
an image generating step for generating an image;
an image correction processing step for performing image correction processing on the image generated at the image generating step to thereby generate a corrected image; and
a display step for displaying the corrected image generated at the image correction processing step on a display screen,
said imaging method including a control step for outputting a control signal for displaying at the display step the image generated at the image generating step,
wherein at the display step, the corrected image displayed on the display screen is displayed by switching to the image generated at the image generating step when the control signal outputted at the control step is received.

12. The imaging method according to claim 11, further comprising a storage step for storing the image generated at the image generating step,
wherein at the display step, the image stored at the storage step is displayed when the control signal outputted at the control step is received.

13. The imaging method according to claim 12, further comprising an operation step for inputting, by operation of an operator, a command for displaying, at the display step, the image generated at the image generating step,
wherein at the control step, the control signal is outputted based on the command inputted by the operator at the operating step.

14. The imaging method according to claim 13, wherein at the image generating step, an RF pulse is transmitted to a subject in a static magnetic field space, and a tomographic image about a tomographic plane of the subject is generated as the image, based on a magnetic resonance signal generated at the subject to which the RF pulse is transmitted.

15. The imaging method according to claim 14, wherein at the image generating step, reference scans and an actual scan are executed.

16. The imaging method according to claim 15, wherein at the image generating step, the reference scans comprises a first reference scan, a second reference scan and a third reference scan,
in the first reference scan, a first RF coil corresponding to a body coil transmits an RF pulse of a first flip angle to an imaging area of the subject, and the first RF coil receives a magnetic resonance signal generated in the imaging area therein,
in the second reference scan, the first RF coil transmits an RF pulse of the first flip angle to the imaging area of the subject, and a second RF coil corresponding to a phased array coil receives a magnetic resonance signal generated in the imaging area therein,
in the third reference scan, the first RF coil transmits an RF pulse of a second flip angle different from the first flip angle to the imaging area of the subject, and the first RF coil receives a magnetic resonance signal generated in the imaging area therein.

17. The imaging method according to claim 16, wherein at the image generating step, a first reference image is generated from the first reference scan and a second reference image is generated from the second reference scan, and a reception sensitivity distribution is obtained based on the first reference image and the second reference image.

18. The imaging method according to claim 17, wherein at the image generating step, a third reference image is generated from the third reference scan, and a transmission sensitivity distribution is obtained based on the first reference image and the second reference image.

19. The imaging method according to claim 18, wherein at the image correction processing step, threshold processing on the transmission sensitivity distribution is executed.

20. The imaging method according to claim 19, wherein at the image correction processing step, the corrected image AIc(x, y) is obtained based on the following equation that includes the reception sensitivity distribution S(x, y), the transmission sensitivity distribution fh(x, y) subsequent to the threshold processing and an actual scan image AI(x, y) that is generated from the actual scan.
AI_{c}(x, y)=AI(x, y)×fh(x, y)×s(x, y)
